(19) United States
(12) Patent Application Publication
(10) Pub. No.: US 2009/0005717 A1
(43) Pub. Date: Jan. 1, 2009

(54) Ankle Joint Bandage

(76) Inventor: Klaus-Dieter Brzank, Meinerzhagen (DE)

Correspondence Address: CLARK & BRODY, 1090 VERMONT AVENUE, NW, SUITE 250, WASHINGTON, DC, 20005, US

(21) Appl. No.: 12/224,381

(22) PCT Filed: Feb. 26, 2007
(86) PCT No.: PCT/EP2007/001617

§ 371 Date: Aug. 26, 2008

(30) Foreign Application Priority Data
Feb. 27, 2006 (DE) 20 2006 003 245.2

(51) Int. Cl. A61F 13/06; A61L 15/00

(52) U.S. Cl. 602/65; 602/63; 602/75

(57) ABSTRACT

An ankle bandage (1) comprises a sock-like or stocking-like anatomically shaped base body (2) made of an extensible textile. Said base body extends between at least a lower leg portion (3) and a foot portion (4) and comprises a pad (5) in at least one malleolar region. To attain both simplicity of use and optimal support of the foot's capsular ligament system at the ankle, the ankle bandage's pad (5) is shaped as a 3D knit in the base body. In an additional or alternative feature, a pronation restraint (6) fitted with an extensible textile ply is laterally mounted on the bandage. The invention also provides that the bandage comprise at least one pad element (9) configured in the region respectively in the course of the peroneal group's tendons and extending in L-shaped manner from the lower leg portion (3) into the foot portion (4) around the malleolar region on the side of the heel respectively on the side of the foot sole.

The present invention relates to an ankle joint bandage comprising an anatomically shaped sock-like or stocking-like base body running at least between a lower leg portion and a foot portion and made of an extensible textile. In particular the present invention relates to an ankle bandage which, depending on applicability respectively medical indication, offers further features illustratively as bandage reinforcements acting as to medical pads or pronation restraints.

The ankle joint is an essential part of the support and moving system of the human body. Especially in sports, it is exposed to high impact and compression loads. When excessively stressed, the soft parts may be injured, for instance by supination traumas due to excessive ligament stretching. Also bending the foot beyond the lateral foot edge may lead to ankle injury. Damages of various gravity are incurred. Accordingly proper ankle function presumes a functional capsular ligament system.

Basically a distinction is made between the upper and lower ankle joints. The upper ankle joint acts as a hinge and allows only stretching or bending movements. The shin bone called tibia and the calf bone called fibula enclose like tongs the ankle bone (trochlea tali) and constitute the malleolar fork. The inner and outer collateral ligament contribute additional stability/strength, the ligaments assuming a fan geometry as a result of which, depending on the joint position, always part of them shall be tensioned. The lower ankle joint allows rotational movements about an oblique axis running from the lateral rear to the medial fore upward, admitting thereby foot pronation and supination. The term pronation is to be construed as a forward and outward rotation, whereas supination is understood as an inward foot rotation.

In sports medicine, after knee injuries, the second most frequent injury is to the ankle. Bandages are used to protect the ankle, to prevent injuries, for protection and reinforcement, and to maintain a sporting lifestyle, further for ailments and post-trauma treatment, such bandages coming in diverse designs. Illustratively there are sock-like and stocking-like bandages enclosing the medial and lateral malleolus of the lower-leg bones using a medical silicone pad, thereby enhancing blood flow and averting congestion as well as supporting the upper ankle joint.

Other designs of ankle bandages include so-called pronation restraints which preferably are configured externally to the foot to avoid food supination and to assure healing an injured capsular ligament system. Also foot joint bandages may comprise medical pads at different foot zones, such pads most of the time being in the form of reinforced textile tissues in the bandage and applying pressure on the relevant foot area, elicitating a massaging effect, or they are used for their support. Most of such medical pads are in the form of silicone inserts which are integrated as molded elements into the basic bandage body and of which the contour and the thickness match the anatomy of the particular foot area.

The German patent document DE 41 04 930 C2 discloses a tubular bandage laterally supporting the ankle in a moderate way and acting in stabilizing manner on the capsular ligament system while simultaneously padding the critical zones of Bisgaard's Kulisse (slide) to attain uniform compression, an intermitted compression distribution caused by a movement applied on the ankle soft parts around the medial and lateral malleolus and deep transverse friction being exerted on the attachment point of the Achilles tendon. Specially designed pads are used for padding. These pads are made in molded form, of a soft or mechanically compliant material, with a lateral, rounded recess to receive bone protrusions, a bar-shaped frictional core made of a hard, incompressible material being configured within the soft or mechanically compliant one.

The German patent document DE 43 18 791 C2 discloses one use of a pronation restraint. The ankle bandage described therein comprises a toe zone made of a tear and tension proof material running straight over and above the ankle and on the inside of a reinforcing insert running in the same direction. Moreover an elastic strap is provided to keep both the pronation restraint and the reinforcing/rigidifying insert in place and is configured in the heel zone of the foot portion and from there it loops the foot instep and the ankle and by its end, following looping the ankle joint, it is affixed to the strap where same encloses the instep.

The German patent document 200 05 742 U1 discloses an ankle bandage comprising a stocking running over the metatarsus and the ankle and an affixation strap adjoining same underneath the foot sole while running in the direction of the foot's outside to enclose the lower leg, the affixation strap running along its looping path over the foot outside and over the foot instep initially as an elastic strap and then, at least completely looping the lower leg at its end position, being a non-stretch strap fitted at its very end with a velcro connection element which can be affixed to a mating velcro element in the ankle bandage.

A similar bandage comprising a bandage material is known from the German patent document 38 38 582 A1, which discloses an ankle-covering, elastic anklebone sock made of an elastic knit, the shape and size of said bandage anatomically matching the ankle to be bandaged, comprising a U-shaped stirrup insert integrated into the anklebone sock and in the form of an antagonistic loop, said insert spirally enclosed by anchor laid around the anklebone, said anchor being an inelastic looping strip with its looping turns spaced from each other and beginning and ending on the outer leg of the stirrup insert.

Regarding an external silicone pad, the German patent document DE 103 05 277 discloses a foot bandage comprising especially in the lateral area of the Achilles tendon a sock-like tube made of an elastic textile running over a lower leg zone, the adjoining heel-ankle zone and the middle foot region, said sock being fitted at each side of the Achilles tendon in the lower leg zone with an elastic pad. The pad situated on the inner side of the tube is fitted with an inner extension element running in the tube's foot arch zone. These silicone pads and their extension elements are approximately lenticular in cross-section, the inside areas comprising naps to improve massaging.

The known ankle bandages incur the drawback of being frequently made entirely of an elastic material, their stabilizing properties foremost being restricted to the prioceptive factor which relates to the body's self-awareness. Frequently these bandages also are multi-part assemblies and entail complex application to the ankle joint, usually requiring much practice and related expertise on the part of the patient.

The comfort of wear of known bandages is frequently limited in that the physiologically effective bandage features such as the pronation restraints, the pads over the malleolus and the outer silicone pads are fairly bulky, causing pain in particular when slipping into shoes and the like and possibly adversely affecting the healing of the capsular ligament system. Also the known ankle bandages do not provide meaningful combinations of several devices supporting the healing of the capsular ligament system, as a result of which at best there is available only a partial positive effect on a limited foot region.

Accordingly it is the objective of the present invention to overcome the drawbacks of the state of the art and to create an ankle bandage allowing easy applicability and always optimally supporting the foot's capsular ligament system in the region of the ankle.

The objective of the present invention is attained by an ankle bandage defined in the preambles of claim 1, claim 9 and claim 15 together with the pertinent features of the invention. Further embodiment modes of the present invention are disclosed in the dependent claims.

As regards an ankle bandage fitted with an anatomically shaped sock-like or stocking-like base body extending at least between a portion of the lower leg and a foot portion and made of an extensible textile, where the bandage is fitted with a pad across at least the area of one ankle, the present invention provides that said pad shall be a 3D knit in said base body.

The invention is based on the insight that a pad shall be formed above the ankle region not in the form a hard molded body but that instead it shall be directly constituted in the textile. This feature offers the substantial advantage that for instance a silicone pad—serving to spread the pressure and relieving the stress on the bone projections—may be eliminated. In this manner more space is left within the shoe and the shoe narrowness is eliminated, comfort being enhanced.

Appropriately the base body's extensible textile shall undergo at the transition into the 3D knit forming the pad a change in texture causing an outward extensibility larger than that of the base body. This 3D knit feature of the pad above the ankle reduces compression, said 3D knit per se assuming the function elsewhere implemented by the pad. Moreover the thickness of the peri-malleolar silicone pad may be reduced.

In its stretched state, the 3D knit constitutes dome-shaped protrusions in the lateral direction in the malleolar region, said protrusions enclosing bone protrusions. The extensible textile comprises a rubbery woven portion, the said texture change of the extensible textile at the transition from the base body into the 3D knit causing a reduction of the rubbery woven portion.

The extensible textile—which also constitutes the base body of the ankle bandage—merges integrally into the 3D knit forming the pad, the textile undergoing only a change in texture. This change in texture applies in particular to a reduced portion of the rubbery fabric in the pad, said fabric assuring elasticity and hence applies the compression on the foot. Because of the reduced portion of the rubbery fabric, the compression is reduced simultaneously, that is the pressure on the malleolar region. In the stressed condition therefore the 3D knit will rise from the plane of the textile in a third dimension, however no significant increase in compression will result.

In another advantageous embodiment of the present invention, the rubbery fabric portion of the extensible textile is prestressed, this prestressing being less in the zone of the 3D knit than in the base body's textile, especially at the transition, in other words, the rubbery fabric portion's prestressing is less at the edge of the 3D knit, “prestressing” denoting that longitudinal tension in the rubbery fabric portion when the textile is unstressed.

The pad-constituting 3D knit is essentially circular, the diameter of the circular 3D knit in its unstressed state being approximately 15 to 35 mm, preferably about 20 to 30 mm and in especially preferred manner about 25 mm. The size of the resulting pad substantially encloses the malleolar region, the stated diameter when the 3D knit is stretched being enlargeable by as much as 50%, in some instances even as much as 100%. Depending on the design of the 3D knit respectively the required shape of the pad, the 3D knit in the unstressed state also may be elliptical, the longitudinal direction of the ellipse corresponding to the longitudinal direction of the lower leg portion of the ankle bandage.

In the relaxed state the height of the lower leg portion from the upper edge to the lower heel edge will be about 200-220 mm, the pad-forming 3D knit being about 100-110 mm from the upper lower leg portion relative to the pad center. When the ankle bandage is plane and unstressed, its width is about 90-110 mm, the pad-forming 3D knit being a distance of about 30-40 mm from the front-foot side edge in the lower leg portion of the base body relative to the pad center.

Advantageously the pad-forming 3D knit is constituted in the extensible textile of the base body in the malleolar region at the outside and/or at the inside of the ankle. As a result the pad extends both across the lateral malleolus and the medial one. However other designs of ankle bandages of the present invention are conceivable which comprise only one pad at the lateral malleolus or at the medial one.

Another feature substantially improving the present invention provides that the ankle bandage include a pronation restraint to stabilize the foot's pronation position, said restraint being configured laterally on the outside of the bandage and including a non-stretch textile ply. Similar to the case of ankle ortheses, the foot's pronation position is supported thereby not only proprioceptively but additionally also mechanically. This restraint at the same time contributes to limiting the advance of the anklebone and to relieve the stress on the front outer ligament, this feature being due in particular by the external, lateral configuration of the restraint. The restraint is automatically tensioned at every twist/sprain and when the foot, is stretched, because it runs from the lateral zone of its sole, initially diagonally, to above the height of the front zone of the ankle bone. An A contour of the textile also contributed to this effect.

Because the upper ankle joint is especially susceptible when the foot is stretched, a slight elevation of the outer foot edge may provide additional safety. The sole (planta pedis) comprises a lateral edge constituting the lateral termination of the walking surface. The pronation restraint begins approximately above this zone and comprises, in the A form, two restraint strings which converge at about the height of the front ankle bone. Due to the A shape, two cross strings run substantially perpendicularly between the two vertically running main restraints, said trans-verse restraints being configured diagonally between said main restraints.

The A-shaped pronation restraint preferably is about 105-115 mm high as a whole and anatomically adapts to the shape of the foot's outer arch. The width of the vertical main restraints is about 12-18 mm, the non-stretch material of the pronation restraint being fitted with a non-woven surface.

Advantageously the pronation restraint is bonded adhesively to the outside of the base body's extensible material. This feature secures a reliable connection between the extensible and non stretch materials, the planar bonding transmitting uniformly the pronation restraint's tension into the extensible textile of the base body. In addition or alternatively to, the pronation restraint may be sewn on the outside of the base body's extensible material. Moreover the connection of the pronation restraint and the base body's extensible textile may be implemented by textile fusion whereby the two textiles are welded into each other. Again any further kind of connection between the materials of the pronation restraint and the base body is applicable.

Another substantially ameliorating feature of the present invention provides that the bandage shall include at least one pad element configured in the zone or extent of the peroneal group's tendons and running in L-shaped manner from the lower lower leg portion into the foot portion around the malleolar region at the heel side or at the foot's sole side. The muscles of the peroneal group actively secure the pronation position of the foot. Said muscles raise the outer foot edge, stabilize the ankle and in this manner help in averting the very commonplace inversion traumas. This outside pad follows the course of the tendons of the pronators and prestresses them. Similarly to the case of a patella tendon bandage, the tone of the securing muscles can be raised as a result and their responsivity can be affected positively.

The upper and the lower ankle joint constitute one unit and both joints together form a sort of universal joint. This feature makes it possible to safely set the foot in its bent or stretched position (dorsal or plantar flexure) on a ground at different slopes. As regards the lower ankle joint, the outer pad contributes during this process to support and stabilize the calcaneo cuboidal joint which is a partial joint of the lower ankle joint. The outer pad supports this joint in two ways. The silicone pad on the foot's outside buffers direct violent effects and the activation of the peroneal group supports the reliable function of the ligament system in this partial joint. Accordingly the formation of the outer pad that includes the calcaneocuboidal joint is an especially advantageous embodiment mode. The pad element may be in the form of a silicone cushion, though other gel-like or compliant materials are also applicable.

The pad element is advantageously received in a pouch medially subtended in the base body, said pouch being constituted between a second textile ply affixed to the base body and the base body per se. In other words, the silicone cushion is sewn by a textile ply on the inside of the foot, onto the base body and as a result encloses the silicone cushion. There is also the feasibility regarding the pad body to configure it at the ankle joint's outer side and/or at the inside laterally. Variations of this design are conceivable, wherein the bandage encloses a silicone cushion either only on the outside, only on the inside, or on both sides.

Other steps ameliorating further the present invention are defined in the dependent claims and are elucidated below together with the description of a preferred, specific, illustrative embodiment of the invention in relation to the attached drawings.

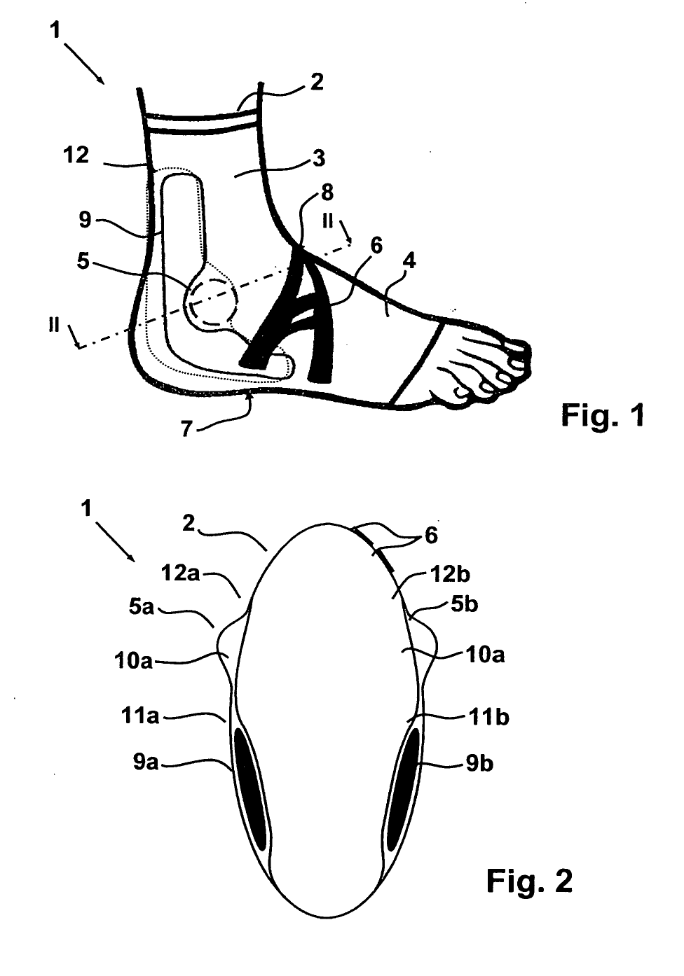

FIG. 1 is a view of a foot clad with an ankle bandage comprising a pad in the malleolar region, a pronation restraint and a pad along the peroneal group's tendons, and

FIG. 2 is a cross-section of an ankle bandage along the sectional plane II-II of FIG. 1.

Both figures relate to an illustrative technical embodiment of the present invention.

FIG. 1 shows a view of an ankle bandage 1 pulled over the right foot. The ankle bandage 1 consists of a sock-like or stocking-like base body 2 which on account of its elasticity adapts to the anatomy of the foot. The bandage is made of one piece and runs from a lower leg portion 3 over the heel and the ankle into a foot portion 4. A pad 5 is indicated in the malleolar region and is in the form of a 3D knit in the base body 2. A pad element 9 runs around the malleoli region and is configured in the zone, respectively along the peroneal group's tendons, extending in L-shape manner from the lower leg portion 3 to the foot portion 4 around the malleoli region on the side of the heel respectively the side of the foot sole. In the region of the pad 5, the pad element 9 is fitted with a recess so that these two zones shall not overlap. In this manner the pad element 9 encloses the malleolar region but does not run over the malleolar bone.

The pad element 9 is designed as a silicon cushion configured medially at the base body 2 as a result of which the extensible textile of the base body 2 comprises an outside, continuous structure. The affixation of the pad element 9 to the base body 2 is implemented by a textile ply which in accordance with the course of the seam 12 is sewn at its inside to the base body 2. A pronation restraint 6 is mounted laterally on the bandage and is bonded by an adhesive on the textile of the base body 2. The basic shape of the pronation restraint 6 is an A and the restraint is made of a non-stretch, tension proof and tear proof material. The pronation restraint 6 runs diagonally from the lateral zone of the sole 7 to approximately above the height of the front zone of the ankle bone 8. The ankle bone 8—in this instance not visible—describes the region within which is placed the lace knot of a low shoe. Because of said A shape, two main restraints converge and essentially diverge like an A in the direction of the foot sole. Two smaller diagonal restraints are integrated between the two main restraints, all restraints being made of an integral, non-stretch textile ply. The outside of the textile ply is fitted with a slipper/felt-like surface to minimize friction for instance within a shoe.

FIG. 2 is a cross-section along the sectional plane II-II indicated in FIG. 1. FIG. 2 shows the ankle bandage 1 in cross-section, so that the base body 2 is shown in contour in the sectional plane. Depending on the shape imparted to the malleolar region, the malleoli are enclosed by a pad 5, both the lateral malleolus and the medial malleolus being enclosed by a pad 5a, 5b. Said pads are shaped as a 3D knit in the extensible textile of the base body 2 and comprises no external molded element or the like. A second textile ply 10 is stitched by means of a seam 12 on the medial side of the base body 2, both inside and out, in order to subtend a pouch 11 within the base body 2. Pad bodies designed as silicone cushions are inserted into said pouches 11a, 11b. Said silicone cushions are cross-sectionally lenticular and foremost support the peroneal group's muscles. The configuration of the pad bodies 9 designed as external pads follows the course of the tendons of the peroneal group's pronators which thus are prestressed. Accordingly the shape of the pad element 9 is adapted to the peroneal group's tendons.

The implementation of the present invention is not restricted to the above preferred illustrative embodiment mode. Instead a plurality of variations may be conceived which fall under the umbrellas of the present invention defined by its solution.

### LIST OF REFERENCES

1 ankle bandage
2 base body
3 lower leg portion
4 foot portion
5 pad
6 pronation restraint
7 foot sole
8 ankle bone
9 pad element
10 textile ply
11 pouch
12 seam

1. An ankle bandage (1) comprising an anatomically adapting sock-like or stocking-like base body (2) running at least from a lower leg portion (3) to a foot portion (4) and being made of a extensible material, said bandage being fitted in at least one malleolus/malleoli region with a pad (5) characterized in that the minimum of one pad (5) is in the form of a 3D knit constituted in the base body.

2. Ankle bandage (1) as claimed in claim 1, characterized in that the extensibility of the base body (2) comprises a change in texture constituting a 3D knit at the transition into the pad (5), said change in texture implementing a laterally outward extensibility larger than the extensibility of the textile material of the base body (2).

3. Ankle bandage (1) as claimed in claim 1, characterized in that the extensible material includes a rubbery fabric portion.

4. Ankle bandage (1) as claimed in claim 3, characterized in that the change in texture of the extensible material includes a reduction of the rubbery fabric portion at the transition from the base body (2) into the 3D knit.

5. Ankle bandage (1) as claimed in claim 3, characterized in that the rubbery fabric portion of the extensible material is prestressed.

6. Ankle bandage (1) as claimed in claim 5, characterized in that the region of the 3D knit is prestressed less than is the extensible material of the base body (2).

7. Ankle bandage (1) as claimed in claim 1, characterized in the 3D knit constituting the pad (5) is circular, the diameter of the circular 3 D knit in its unstretched state being about 15 to 35 mm, preferably about 20 to 30 mm, and in especially preferred manner about 25 mm.

8. Ankle bandage (1) as claimed in claim 1, characterized in that the 3D knit constituting the pad (5) is constituted in the extensible textile of the base body (2) in the malleolar region at the outside and/or at the inside of the ankle.

9. Ankle bandage (1) comprising an anatomically shaped sock-like or stocking-like base body (2) extending at least between a lower leg portion (3) and a foot portion (4) and made of an extensible textile, said bandage further comprising a pronation restraint (6) stabilizing the foot's pronation position, characterized in that the pronation restraint (6) is laterally configured at the bandage and includes a non-stretch textile ply.

10. Ankle bandage (1) as claimed in claim 9, characterized in that the textile ply's contour is A-shaped.

11. Ankle bandage (1) as claimed in claim 9, characterized in that the pronation restraint (6) runs initially diagonally from the lateral zone of the foot sole (7) as far as approximately above the height of the front zone of the ankle bone (8).

12. Ankle bandage (1) as claimed in claim 9, characterized in that the pronation restraint (6) is adhesively bonded to the outside of the extensible textile of the base body (2).

13. Ankle bandage (1) as claimed in claim 9, characterized in that the pronation restraint (6) is sewn to the outside of the extensible textile of the base body (2).

14. Ankle bandage (1) as claimed in claim 9, characterized in that, using a textile fusion method, the pronation restraint (6) is welded to the outside of the extensible textile of the base body (2).

15. Ankle bandage (1) comprising an anatomically shaped sock-like or stocking-like base body (2) running at least between a lower leg portion (3) and a foot portion (4) and made of an extensible textile, characterized in that the bandage comprises at least one pad element (9) configured in the region respectively along the course of the peroneal group's tendons and extending in L-shaped manner from the lower leg portion (3) into the foot portion (4) around the malleolar region at the heel side respectively the foot sole side.

16. Ankle bandage (1) as claimed in claim 15, characterized in that the pad element (9) is a silicone cushion.

17. Ankle bandage (1) as claimed in claim 15, characterized in that the pad element (9) is received is a pouch (11) medially configured at the base body (2), said pouch (11) being constituted between the base body (2) and a second textile ply (10) configured on said base body (2).

18. Ankle joint (1) as claimed in claim 17, characterized in that the pouch (11) medially constituted at the base body (2) is sewn by a seam (12), of which the location corresponds to the contour of the pouch (11) to the base body (2).

19. Ankle bandage (1) as claimed in claim 15, characterized in that the pad element (9) is configured at the outside and/or at the inside of the ankle.
